(19) United States
(12) Patent Application Publication
(10) Pub. No.: US 2019/0322830 A1
(43) Pub. Date: Oct. 24, 2019

(54) POROUS MATERIAL, GAS SENSOR, AND METHOD FOR PRODUCING POROUS MATERIAL

(71) Applicant: SUMITOMO ELECTRIC FINE POLYMER, INC., Sennan-gun, Osaka (JP)

(72) Inventors: Fumihiro HAYASHI, Sennan-gun, Osaka (JP); Atsushi UNO, Sennan-gun, Osaka (JP); Yoshimasa SUZUKI, Sennan-gun, Osaka (JP); Yasuhiko MUROYA, Sennan-gun, Osaka (JP); Takayuki USUI, Sennan-gun, Osaka (JP)

(73) Assignee: SUMITOMO ELECTRIC FINE POLYMER, INC., Sennan-gun, Osaka (JP)

(21) Appl. No.: 16/470,640

(22) PCT Filed: Jul. 24, 2017
(86) PCT No.: PCT/JP2017/026651

§ 371 Date: Jun. 18, 2019

(30) Foreign Application Priority Data
Dec. 22, 2016 (JP) 2016-249895

(51) Int. Cl. C08J 9/00; G01N 33/00

(52) U.S. Cl. CPC C08J 9/0061; C08J 2427/18; C08J 2327/18; G01N 33/0027

(57) ABSTRACT

A porous material according to an embodiment of the present invention is a porous material having a large number of fibrous skeletons containing polytetrafluoroethylene as a main component, in which another fluororesin is evenly present on outer peripheral surfaces of fibers of the large number of fibrous skeletons, and the other fluororesin is a tetrafluoroethylene/perfluorodioxole copolymer, a tetrafluoroethylene/perfluoromethyl vinyl ether copolymer, a tetrafluoroethylene/perfluoroethyl vinyl ether copolymer, a tetrafluoroethylene/perfluoropropyl vinyl ether copolymer, or a combination of these.

### TECHNICAL FIELD

The present invention relates to a porous material, a gas sensor, and a method for producing a porous material.

The present application claims priority from Japanese Patent Application No. 2016-249895 filed on Dec. 22, 2016, and the entire contents of the Japanese patent application are incorporated herein by reference.

### BACKGROUND ART

In recent years, gas sensors have been used in order to measure, for example, a concentration of oxygen contained in exhaust gas of automobiles. Such a gas sensor has a vent portion for introducing external gas. For the vent portion, gas permeability and high heat resistance sufficient for automotive exhaust gas are required. When oil used in, for example, the maintenance of an automobile is impregnated into or adheres to the vent portion, holes of the vent portion are clogged, which may cause a decrease in gas permeability. Therefore, it is desirable for the vent portion to have good oil repellency in addition to gas permeability and heat resistance.

As a sheet used in such a vent portion, for example, a porous sheet has been proposed in which a surface of a porous substrate including stretched polytetrafluoroethylene is coated with a copolymer of tetrafluoroethylene and a perfluoroalkyl vinyl ether (refer to Japanese Unexamined Patent Application Publication (Translation of PCT Application) No. 2005-535877).

The porous sheet described in the above patent application publication includes a porous substrate including stretched polytetrafluoroethylene and thus has high heat resistance. It is further disclosed that, in this porous sheet, oil repellency on the surface side of the porous substrate can be enhanced by coating the surface with a copolymer of tetrafluoroethylene and a perfluoroalkyl vinyl ether.

Regarding a technique for enhancing oil repellency of a stretched porous polytetrafluoroethylene membrane, a waterproof air-permeable filter has also been proposed in which a stretched porous polytetrafluoroethylene membrane is coated with a copolymer of perfluoro(2,2-dimethyl-1,3-dioxole) and tetrafluoroethylene (refer to Japanese Unexamined Patent Application Publication No. 5-320255).

### CITATION LIST

### Patent Literature

PTL 1: Japanese Unexamined Patent Application Publication (Translation of PCT Application) No. 2005-535877
PTL 2: Japanese Unexamined Patent Application Publication No. 5-320255

### SUMMARY OF INVENTION

A porous material according to an embodiment of the present invention is a porous material having a large number of fibrous skeletons containing polytetrafluoroethylene as a main component, in which another fluororesin is evenly present on outer peripheral surfaces of fibers of the large number of fibrous skeletons, and the other fluororesin is a tetrafluoroethylene/perfluorodioxole copolymer, a tetrafluoroethylene/perfluoromethyl vinyl ether copolymer, a tetrafluoroethylene/perfluoroethyl vinyl ether copolymer, a tetrafluoroethylene/perfluoropropyl vinyl ether copolymer, or a combination of these.

A gas sensor according to another embodiment of the present invention includes the porous material in a vent portion.

A method for producing a porous material according to another embodiment of the present invention is a method for producing a porous material having a large number of fibrous skeletons containing polytetrafluoroethylene as a main component, the method including a mixing step of mixing a polytetrafluoroethylene powder, another fluororesin, and a fluorine-containing organic solvent, and an extrusion step of extruding a resin composition obtained by the mixing, in which the other fluororesin is a tetrafluoroethylene/perfluorodioxole copolymer, a tetrafluoroethylene/perfluoromethyl vinyl ether copolymer, a tetrafluoroethylene/perfluoroethyl vinyl ether copolymer, a tetrafluoroethylene/perfluoropropyl vinyl ether copolymer, or a combination of these.

### BRIEF DESCRIPTION OF DRAWINGS

FIG. 1 is a schematic perspective view illustrating a porous material according to an embodiment of the present invention.

FIG. 2 is a schematic view illustrating a gas sensor including the porous material in FIG. 1.

### DESCRIPTION OF EMBODIMENTS

### Problems to be Solved by Present Disclosure

In the existing porous sheet and waterproof air-permeable filter described in the above patent application publications, oil repellency on the surface sides of the porous substrate and the stretched porous polytetrafluoroethylene membrane is partially enhanced by coating. Accordingly, oil repellency may become insufficient when the coating peels off due to, for example, damage on the surface sides of the porous substrate and the stretched porous polytetrafluoroethylene membrane. Furthermore, in the porous sheet and the waterproof air-permeable filter, when the amount of a layer of coating is increased so as to address the damage on the surface sides of the porous substrate and the stretched porous polytetrafluoroethylene membrane to a certain degree, this coating may clog pores of the porous substrate and the stretched porous polytetrafluoroethylene membrane. Accordingly, it is difficult to sufficiently enhance both gas permeability and oil repellency of the porous sheet and the waterproof air-permeable filter.

The present invention has been made on the basis of the circumstances described above. An object of the present invention is to provide a porous material and a gas sensor that have high gas permeability and oil repellency, and a method for producing the porous material.

### Advantageous Effects of Present Disclosure

The porous material and the gas sensor of the present invention have high gas permeability and oil repellency. According to the method for producing a porous material of the present invention, a porous material having high gas permeability and oil repellency can be produced.

### DESCRIPTION OF EMBODIMENTS OF PRESENT INVENTION

First, embodiments of the present invention will be listed and described.

A porous material according to an embodiment of the present invention is a porous material having a large number of fibrous skeletons containing polytetrafluoroethylene (PTFE) as a main component, in which another fluororesin is evenly present on outer peripheral surfaces of fibers of the large number of fibrous skeletons, and the other fluororesin is a tetrafluoroethylene/perfluorodioxole copolymer (TFE/PDD), a tetrafluoroethylene/perfluoromethyl vinyl ether copolymer (TFE/MFA), a tetrafluoroethylene/perfluoroethyl vinyl ether copolymer (TFE/EFA), a tetrafluoroethylene/perfluoropropyl vinyl ether copolymer (TFE/PFA), or a combination of these.

The porous material has a large number of fibrous skeletons containing PTFE as a main component, and a plurality of pores are formed in regions surrounded by the fibrous skeletons. Since the other fluororesin is evenly present on outer peripheral surfaces of fibers of the fibrous skeletons, the porous material has high gas permeability and oil repellency. That is, since the other fluororesin is also present on outer peripheral surfaces of fibers located inside the porous material, a decrease in oil repellency of the porous material can be suppressed even if the surface side is damaged. Furthermore, in the porous material, since the other fluororesin is evenly present on outer peripheral surfaces of fibers, the possibility of clogging of the pores due to partial localization of this fluororesin is low.

A content of the other fluororesin relative to 100 parts by mass of the PTFE is preferably 0.08 parts by mass or more and 2.0 parts by mass or less. When the content of the other fluororesin relative to 100 parts by mass of the PTFE is within the above range, oil repellency can be sufficiently enhanced while the amount of the other fluororesin used is kept relatively low.

The porous material preferably has a porosity of 30% by volume or more and 80% by volume or less. When the porosity is within the above range, gas permeability can be sufficiently enhanced while a decrease in strength is suppressed.

The other fluororesin is preferably also present inside the fibers of the large number of fibrous skeletons. When the other fluororesin is also present inside the fibers of the large number of fibrous skeletons, separation of the other fluororesin from the fibers is suppressed. Accordingly, a decrease in oil repellency due to separation of the other fluororesin can be suppressed.

A gas sensor according to another embodiment of the present invention includes the porous material in a vent portion. Since the gas sensor includes the porous material in a vent portion, both gas permeability and oil repellency of the vent portion can be sufficiently enhanced.

A method for producing a porous material according to another embodiment of the present invention is a method for producing a porous material having a large number of fibrous skeletons containing PTFE as a main component, the method including a mixing step of mixing a PTFE powder, another fluororesin, and a fluorine-containing organic solvent, and an extrusion step of extruding a resin composition obtained by the mixing, in which the other fluororesin is TFE/PDD, TFE/MFA, TFE/EFA, TFE/PFA, or a combination of these.

The method for producing a porous material can produce the porous material which has a large number of fibrous skeletons containing PTFE as a main component and in which the other fluororesin is evenly present on outer peripheral surfaces of fibers of the fibrous skeletons, the porous material thereby having high gas permeability and oil repellency.

In the present invention, the term “main component” refers to a component that has the highest content, and for example, a component having a content of 50% by mass or more. The term “porosity” refers to a percentage of a total volume of pores in a porous material having the pores to a volume of this porous material. This porosity can be calculated by (V1−V0)/V1×100 where V0 represents a solid volume of a porous material having pores, the solid volume being calculated from the mass and density of a solid portion of the porous material, and V1 represents an apparent volume of the porous material having pores, the apparent volume including the volume of the pores of the porous material.

### DETAILS OF EMBODIMENTS OF PRESENT INVENTION

Hereinafter, a porous material, a gas sensor, and a method for producing a porous material according to embodiments of the present invention will be described with appropriate reference to the drawings.

### [Porous Material]

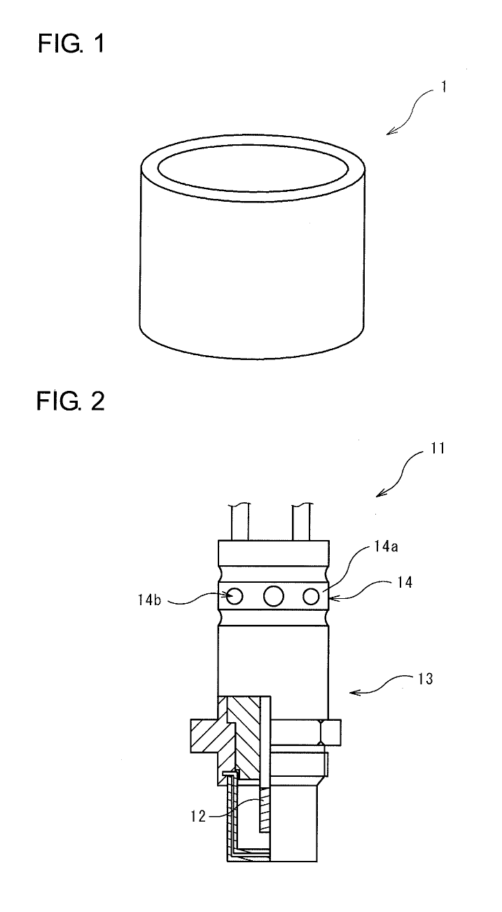

A porous material 1 in FIG. 1 has a large number of fibrous skeletons containing PTFE as a main component. In the porous material 1, another fluororesin is evenly present on outer peripheral surfaces of fibers of the large number of fibrous skeletons, and the other fluororesin is TFE/PDD, TFE/MFA, TFE/EFA, TFE/PFA, or a combination of these. The other fluororesin is preferably soluble in organic solvents. In this case, the lower limit of a copolymerization ratio of PDD, MFA, EFA, and PFA in the other fluororesin is preferably 40% by mole, more preferably 60% by mole, and still more preferably 80% by mole relative to the total amount of the copolymer.

The porous material 1 has a large number of fibrous skeletons containing PTFE as a main component, and this PTFE has good heat resistance, chemical stability, weather resistance, incombustibility, strength, and the like. The porous material 1 has a plurality of pores that are formed in regions surrounded by the large number of fibrous skeletons. In the porous material 1, the other fluororesin is evenly present on outer peripheral surfaces of fibers of the fibrous skeletons. In other words, since the other fluororesin is not one that is impregnated or stacked in the form of a layer on the surface side of the porous material 1 by coating or the like, the porous material 1 has high gas permeability and oil repellency. That is, since the other fluororesin is also present on the outer peripheral surfaces of fibers located inside the porous material 1, a decrease in oil repellency of the porous material 1 can be suppressed even if the surface side of the porous material 1 is damaged. Furthermore, in the porous material 1, since the other fluororesin is evenly present on outer peripheral surfaces of fibers, the possibility of clogging of the pores due to partial localization of this fluororesin is low.

The porous material 1 is in the form of a tube or a sheet (an example of the tubular form is shown in FIG. 1). The porous material 1 has flexibility. The porous material 1 is a single-layer body having a large number of fibrous skeletons containing PTFE as a main component. The fibrous skeletons have a network structure in which particle aggregates (secondary particles) called nodes are connected together with fibrous portions called fibrils therebetween. Gaps between fibrils and between a fibril and a node form pores in the porous material 1.

The lower limit of a content of the PTFE in the porous material 1 is preferably 90% by mass, more preferably 95% by mass, and still more preferably 98% by mass. When the content of the PTFE is less than the lower limit, the porous material 1 may have insufficient heat resistance.

The lower limit of an average thickness of the porous material 1 is preferably 50 μm and more preferably 100 μm. On the other hand, the upper limit of the average thickness is preferably 5 mm and more preferably 3 mm. When the average thickness is smaller than the lower limit, the porous material 1 may have insufficient strength. On the other hand, when the average thickness exceeds the upper limit, gas permeability of the porous material 1 may decrease.

In the porous material 1, another fluororesin composed of TFE/PDD, TFE/MFA, TFE/EFA, TFE/PFA, or a combination of these is evenly present on outer peripheral surfaces of fibers of the fibrous skeletons. The porous material 1 is, for example, an extrusion molded body formed by extrusion-molding a resin composition containing a PTFE powder and the other fluororesin into a tubular or sheet shape. When the fibrous skeletons are formed from particles of the PTFE powder by this extrusion molding, the other fluororesin covers the surface of the PTFE. With this structure, the porous material 1 has both water repellency and oil repellency and can maintain these characteristics for a long time at a high temperature. Examples of the TFE/PDD include Teflon (registered trademark) AF grade such as “AF1600” and “AF2400” (manufactured by Du Pont-Mitsui Fluorochemicals Company, Ltd.) and Algoflon series such as “Algoflon (registered trademark) AD” (manufactured by Solvay Specialty Polymers Japan K.K.).

The lower limit of a content of the other fluororesin relative to 100 parts by mass of the PTFE is preferably 0.08 parts by mass and more preferably 0.1 parts by mass. On the other hand, the upper limit of the content of the other fluororesin relative to 100 parts by mass of the PTFE is preferably 2.0 parts by mass, more preferably 1.0 part by mass, and still more preferably 0.6 parts by mass. When the content is less than the lower limit, oil repellency of the porous material 1 may not sufficiently improve. In contrast, when the content exceeds the upper limit, the production cost of the porous material 1 may unnecessarily increase. In the case of the related art in which a surface of a porous base material containing PTFE as a main component is coated with the other fluororesin, the coating may peel off when the amount of coating is insufficient. Accordingly, in order to achieve sufficient oil repellency, the content of the other fluororesin relative to the PTFE becomes relatively high, and the production cost increases. However, when the amount of coating is increased so as to obtain sufficient oil repellency, pores on the surface side of the porous base material are easily clogged by the other fluororesin. That is, according to the above coating, there is a trade-off relationship between oil repellency and gas permeability. In contrast, in the porous material 1, when the content of the other fluororesin relative to 100 parts by mass of the PTFE is within the above range, sufficient oil repellency can be achieved while the production cost is kept low, and the possibility of clogging of pores with the other fluororesin is low.

The lower limit of a porosity of the porous material 1 is preferably 30% by volume and more preferably 40% by volume. On the other hand, the upper limit of the porosity is preferably 80% by volume and more preferably 70% by volume. When the porosity is less than the lower limit, the porous material 1 may have insufficient gas permeability. On the other hand, when the porosity exceeds the upper limit, the porous material 1 may have insufficient strength. In the case of the related art in which a surface of a porous base material containing PTFE as a main component is coated with the other fluororesin, when the porosity is low, there is a high possibility that pores on the surface side are clogged by the coating. On the other hand, when the porosity is high in the above case of coating, the other fluororesin cannot be sufficiently stacked on the surface side, and sufficient oil repellency may not be obtained. In contrast, in the porous material 1, since the other fluororesin is evenly present on surfaces of the fibers, while sufficient oil repellency is achieved, sufficient gas permeability can be achieved with the porosity in the above range.

In the porous material 1, preferably, the other fluororesin is also present inside fibers of the large number of fibrous skeletons. In the porous material 1, when the fibrous skeletons are formed from particles of a PTFE powder by extrusion molding, the other fluororesin covers the surfaces of the particles of the PTFE powder. Accordingly, in some cases, the other fluororesin may be present inside the fibers in a state in which particles of the PTFE powder are connected together in the form of fibers. Furthermore, the other fluororesin that is present inside the fibers may be partially exposed on the outside of the fibers. In the porous material 1, when the other fluororesin is present inside fibers of the large number of fibrous skeletons in this manner, separation of the other fluororesin from the fibers is suppressed, and thus a decrease in oil repellency due to the separation of the other fluororesin can be suppressed. As a result, the porous material 1 can maintain oil repellency for a long period of time. Furthermore, in the case where the other fluororesin is also present inside fibers of the large number of fibrous skeletons, the effect of oil repellency lasts because a new oil repellent agent is exposed at the surface when damage such as scraping, wear, or a scratch is generated.

The porous material 1 may contain, in addition to the PTFE and the other fluororesin, other additives within a range that does not impair desired effects of the present invention. Examples of the other additives include pigments for coloring, inorganic fillers, metal powders, metal oxide powders, and metal sulfide powders for improving wear resistance, preventing cold flow, or facilitating formation of pores.

### [Production Method]

Next, a method for producing the porous material 1 will be described. The method for producing a porous material includes a mixing step of mixing a PTFE powder, another fluororesin, and a fluorine-containing organic solvent, and an extrusion step of extruding a resin composition obtained by the mixing, in which the other fluororesin is TFE/PDD, TFE/MFA, TFE/EFA, TFE/PFA, or a combination of these.

The method for producing a porous material can easily and reliably produce the porous material 1 which has a large number of fibrous skeletons containing PTFE as a main component and in which the other fluororesin is evenly present on outer peripheral surfaces of fibers of the fibrous skeletons, the porous material 1 thereby having high gas permeability and oil repellency.

### (Mixing Step)

In the mixing step, for example, a solution obtained by mixing the other fluororesin and a fluorine-containing organic solvent is mixed with a PTFE powder to thereby prepare a resin composition in which PTFE and the other fluororesin are uniformly dispersed in the fluorine-containing organic solvent. An average particle size of particles of the PTFE powder can be, for example, 200 nm or more and 300 nm or less. In the method for producing a porous material, since the other fluororesin functions as an extrusion aid in the extrusion step, which will be described below, preferably, another extrusion aid is not mixed from the viewpoint of keeping the production cost low.

Examples of the fluorine-containing organic solvent include heptacosafluorotributylamine, hexafluorobenzene, perfluorooctane, perfluoroheptane, perfluorotriethylamine, perfluorononane, perfluoroethers, 2H,3H-decafluoropentane, 1H,1H,10H,10H-hexadecafluoro-1,10-decanediol, 1H,1H-nonafluoro-1-pentanol, 2,2,3,3,3-pentafluoro-1-propanol, 2,2,3,3,4,4,4-heptafluoro-1-butanol, and methyl heptafluorobutyrate.

The lower limit of a content of the other fluororesin relative to 100 parts by mass of the fluorine-containing organic solvent is preferably 0.02 parts by mass and more preferably 0.06 parts by mass. On the other hand, the upper limit of the content is preferably 2.0 parts by mass and more preferably 0.5 parts by mass. When the content is lower than the lower limit, the production cost of the porous material 1 may unnecessarily increase. In contrast, when the content exceeds the upper limit, it may become difficult to perform extrusion.

In the mixing step, in addition to the PTFE powder, the other fluororesin, and the fluorine-containing organic solvent, the other additives described above may be mixed within a range that does not impair desired effects of the present invention. Furthermore, in order to promote the formation of a porous structure of the porous material, substances that are removed or decomposed by heating, extraction, dissolution, or the like, for example, ammonium chloride, sodium chloride, rubber, and the like may be blended in the mixing step.

### (Extrusion Step)

In the extrusion step, the resin composition mixed in the mixing step is extrusion-molded into a tubular or sheet shape. An extrusion temperature in this extrusion step is, for example, preferably 30° C. or higher and more preferably 50° C. or higher. The extrusion temperature is preferably equal to or lower than the melting point of PTFE.

The method for producing a porous material preferably further includes, after the extrusion step, a stretching step of stretching a tubular or sheet-like extruded body extruded in the extrusion step and a heat treatment step of performing heat treatment after stretching. In the method for producing a porous material, the pores size and the porosity of the porous material obtained can be adjusted by adjusting a stretching temperature and a stretching ratio in the stretching step. Furthermore, in the method for producing a porous material, thermal shrinkage of the extruded body after stretching is suppressed in the heat treatment step, and the porous structure can be reliably maintained. In the method for producing a porous material, the fluorine-containing organic solvent is preferably completely volatilized in the extrusion step or the stretching step.

### (Stretching Step)

The lower limit of the stretching temperature in the stretching step is preferably 200° C. and more preferably 260° C. On the other hand, the upper limit of the stretching temperature is preferably 350° C. and more preferably 300° C. When the stretching temperature is less than the lower limit, the pore size may become excessively large. In contrast, when the stretching temperature exceeds the upper limit, the pore size may become excessively small.

The lower limit of the stretching ratio in a longitudinal direction (an extrusion direction) in the stretching step is preferably 1.5 times and more preferably 1.7 times. On the other hand, the upper limit of the stretching ratio in the longitudinal direction is preferably 7 times and more preferably 5 times. Biaxial stretching may be performed in the stretching step. In this case, the lower limit of the stretching ratio in a transverse direction (a direction perpendicular to the extrusion direction) is preferably 2 times and more preferably 4 times. On the other hand, the upper limit of the stretching ratio in the transverse direction is preferably 40 times and more preferably 20 times. When the stretching ratios in the longitudinal direction and the transverse direction are less than the lower limits, the porous material obtained may have an insufficient porosity. In contrast, when the stretching ratios in the longitudinal direction and the transverse direction exceed the upper limits, cracks may be generated in the porous material obtained, or the size of pores may become unnecessarily large.

### (Heat Treatment Step)

In the heat treatment step, for example, the extruded body after stretching is held in a heating furnace whose temperature is maintained at a temperature equal to or higher than the melting point of the PTFE powder, for example, 350° C. or higher and 550° C. or lower, for about several tens of seconds to several minutes to bake the extruded body.

### [Gas Sensor]

A gas sensor 11 in FIG. 2 includes a sensor element 12 and a casing 13 that houses the sensor element 12. The gas sensor 11 has a vent portion 14 through which gas present in the outside is introduced on the proximal end side of the sensor element 12. The gas sensor 11 includes the porous material 1 in the vent portion 14. Specifically, the vent portion 14 is formed as part of the casing 13 and includes a gas inlet portion 14a having a plurality of gas inlet holes 14b and the porous material 1 disposed on the inner surface side of the gas inlet portion 14a. The porous material 1 is disposed such that the outer circumferential surface of the porous material 1 faces the inner circumferential surface of the gas inlet portion 14a. The gas sensor 11 is disposed, for example, in an engine exhaust passage of an automobile and configured to measure a concentration of oxygen contained in exhaust gas of the automobile.

Since the gas sensor 11 includes the porous material 1 in the vent portion 14, both gas permeability and oil repellency of the vent portion 14 can be sufficiently enhanced.

### Other Embodiments

It is to be understood that the embodiments disclosed herein are only illustrative and non-restrictive in all respects. The scope of the present invention is not limited to the configurations of the embodiments and is defined by the claims described below. The scope of the present invention is intended to cover all the modifications within the meaning and the scope of equivalents of the claims. For example, the porous material is not necessarily provided in a vent portion of a gas sensor and may be provided in a portion other than the vent portion in a gas sensor. The porous material may be used as another member such as a filter for medical applications.

### EXAMPLES

While the present invention will be described in more detail below by way of examples, the present invention is not limited to these examples.

### <Preparation of Samples>

### [No. 1 to No. 5]

Resin compositions prepared by mixing a PTFE powder (“Fluon (registered trademark) CD123E” manufactured by Asahi Glass Co., Ltd.), TFE/PDD (“AF2400” manufactured by Du Pont-Mitsui Fluorochemicals Company, Ltd.) serving the other fluororesin described above, and heptacosafluorotributylamine serving as a fluorine-containing organic solvent were each fcd to a single-screw extruder having a diameter (inner diameter) of 10 mm, extruded into a string shape from a capillary having a die diameter of 2 mm at a cylinder set temperature (extrusion temperature) of 50° C. at an extrusion rate of 60 mm/min, and stretched in the longitudinal direction (extrusion direction) at 270° C. at a ratio of 2 times to prepare samples of No. 1 to No. 5. Table 1 shows contents of a component used in the samples and porosities of the samples.

### [No. 6]

A resin composition prepared by mixing a PTFE powder (“Fluon (registered trademark) CD123E” manufactured by Asahi Glass Co., Ltd.) and solvent naphtha serving as a lubricant aid was extruded under the same conditions as those in No. 1 to No. 5 to prepare a sample of No. 6. Table 1 shows a porosity of the sample.

### [No. 7 and No. 8]

Surfaces of samples that were the same as the sample of No. 6 were coated with a coating liquid in which TFE/PDD (“AF2400” manufactured by Du Pont-Mitsui Fluorochemicals Company, Ltd.) was dispersed in heptacosafluorotributylamine serving as a fluorine-containing organic solvent to prepare samples of No. 7 and No. 8. Table 1 shows contents of a component used in the samples and porosities of the samples.

TABLE 1

| | Resin composition or coating liquid | | | |
|---|---|---|---|---|
| | | Ratio of other fluororesin | Sample | |
| | | relative to 100 parts by | Ratio of other fluororesin | Porosity |
| | Fluorine-containing | mass of fluorine-containing | relative to 100 parts by | [% by |
| | organic solvent | organic solvent | mass of PTFE | volume] |
| No. 1 | Heptacosafluorotributylamine | 0.17 | 0.07 | 65 |
| No. 2 | Heptacosafluorotributylamine | 0.21 | 0.09 | 65 |
| No. 3 | Heptacosafluorotributylamine | 0.52 | 0.22 | 65 |
| No. 4 | Heptacosafluorotributylamine | 1.01 | 0.42 | 65 |
| No. 5 | Heptacosafluorotributylamine | 1.52 | 0.63 | 65 |
| No. 6 | Solvent naphtha | — | — | 65 |
| No. 7 | Heptacosafluorotributylamine | 0.06 | 0.09 | 65 |
| No. 8 | Heptacosafluorotributylamine | 0.26 | 0.42 | 65 |

### <Oil Repellency of Surface>

An impregnation property of ethanol for each of the samples of No. 1 to No. 8 after ethanol was applied to the samples and the samples were maintained at room temperature (25° C.) for three minutes was examined by visual observation. Thus, oil repellency of a surface was evaluated in accordance with the criteria described below. Table 2 shows the evaluation results.
A: Ethanol does not permeate.
B: Ethanol partially permeates.
C: Ethanol permeates.

### <Oil Repellency of the Whole>

The samples of No. 1 to No. 8 were immersed in ethanol and maintained at room temperature (25° C.) for one hour. A weight ratio of each of the samples before and after the immersion was measured. Thus, overall oil repellency of each of the samples was evaluated in accordance with the criteria described below. Table 2 shows the evaluation results.
A: Ethanol does not permeate.
B: Ethanol partially permeates.
C: Ethanol permeates.

TABLE 2

| | Oil repellency of surface | Oil repellency of the whole |
|---|---|---|
| No. 1 | B | B |
| No. 2 | A | A |
| No. 3 | A | A |
| No. 4 | A | A |
| No. 5 | A | A |
| No. 6 | C | C |
| No. 7 | B | C |
| No. 8 | B | C |

### [Evaluation Results]

Table 2 shows that the samples of No. 1 to No. 5 each have good oil repellency of the inside in addition to good oil repellency of the surface. It is found that, in particular, the samples of No. 2 to No. 5, each of which has a higher content of the other fluororesin than No. 1, have good oil repellency of the surface and good oil repellency of the inside. In contrast, regarding the samples of No. 7 and No. 8, it is found that while the surface has a certain degree of oil repellency, the inside has insufficient oil repellency because these samples are prepared by coating of TFE/PDD. Therefore, it is considered that oil repellency of the samples of No. 7 and No. 8 may decrease extremely when, for example, the surface is damaged. In addition, in each of No. 7 and No. 8, the ratio of the fluorine-containing organic solvent used in the preparation of the samples is higher than that in No. 1 to No. 5, which suggests a high production cost. It is found that since the sample of No. 6 does not contain TFE/PDD, both oil repellency of the surface and oil repellency of the whole are insufficient.

### REFERENCE SIGNS LIST

1 porous material
11 gas sensor
12 sensor element
13 casing
14 vent portion
14a gas inlet portion
14b gas inlet hole

1. A porous material comprising a large number of fibrous skeletons containing polytetrafluoroethylene as a main component,
wherein another fluororesin is evenly present on outer peripheral surfaces of fibers of the large number of fibrous skeletons, and
the other fluororesin is a tetrafluoroethylene/perfluorodioxole copolymer, a tetrafluoroethylene/perfluoromethyl vinyl ether copolymer, a tetrafluoroethylene/perfluoroethyl vinyl ether copolymer, a tetrafluoroethylene/perfluoropropyl vinyl ether copolymer, or a combination of these.

2. The porous material according to claim 1, wherein a content of the other fluororesin relative to 100 parts by mass of the polytetrafluoroethylene is 0.08 parts by mass or more and 2.0 parts by mass or less.

3. The porous material according to claim 1, wherein the porous material has a porosity of 30% by volume or more and 80% by volume or less.

4. The porous material according to claim 1, wherein the other fluororesin is also present inside the fibers of the large number of fibrous skeletons.

5. The porous material according to claim 1, wherein the porous material has an average thickness of 50 μm or more and 5 mm or less.

6. A gas sensor comprising the porous material according to claim 1 in a vent portion.

7. A method for producing a porous material having a large number of fibrous skeletons containing polytetrafluoroethylene as a main component, the method comprising:
a mixing step of mixing a polytetrafluoroethylene powder, another fluororesin, and a fluorine-containing organic solvent; and
an extrusion step of extruding a resin composition obtained by the mixing,
wherein the other fluororesin is a tetrafluoroethylene/perfluorodioxole copolymer, a tetrafluoroethylene/perfluoromethyl vinyl ether copolymer, a tetrafluoroethylene/perfluoroethyl vinyl ether copolymer, a tetrafluoroethylene/perfluoropropyl vinyl ether copolymer, or a combination of these.

8. The method for producing a porous material according to claim 7, wherein in the mixing step, a content of the other fluororesin relative to 100 parts by mass of the fluorine-containing organic solvent is 0.02 parts by mass or more and 2.0 parts by mass or less.
